(19) United States
(12) Patent Application Publication
(10) Pub. No.: US 2012/0271324 A1
(43) Pub. Date: Oct. 25, 2012

(54) METAL SUBSTRATE HEATER

(76) Inventors: John V. Yannone, Seabrook, NH (US); Christopher Petroff, Groton, MA (US); Keith Boudreau, Beverly, MA (US)

(21) Appl. No.: 13/392,826

(22) PCT Filed: Aug. 26, 2010
(86) PCT No.: PCT/US10/46809

§ 371 Date: Jun. 14, 2012

Related U.S. Application Data
(60) Provisional application No. 61/237,617, filed on Aug. 27, 2009.

(30) Foreign Application Priority Data
Oct. 12, 2009 (FR) 0957122

(51) Int. Cl. A61B 17/04; H05B 3/02

(52) U.S. Cl. 606/144; 219/538

(57) ABSTRACT

A heating element with dimensions less than a centimetre according to an embodiment of the present invention includes a metallic substrate (1) which includes a material having a thermal conductivity greater than 5 W/m·k and an ultimate tensile strength measured at ambient temperature greater than 400 MPa and which includes a working face suitable to be placed in contact with a medium or an element to be heated, a conductive layer (3) which is supported by a face of the substrate opposite the working face and which forms a circuit comprising two power supply elements (A) linked by a thin resistive element (R) and adapted to be connected to a current source, and a dielectric layer (2) interposed between the conductive layer (3) and the metallic substrate (1).

This application claims the benefit of U.S. Provisional Patent Application Ser. No. 61/237,617, filed Aug. 27, 2009, and French Patent Application No. 09 57122, filed Oct. 12, 2009, which are incorporated herein by reference.

Embodiments of the present invention relate to the technical field of small heating elements, namely those with a size less than a centimeter, or less than a half centimeter. In one application, embodiments of the invention relate to the use of such heating elements in medical devices, such as surgical instruments for closing sutures by welding portions of thermofusible suture strands.

In the context of such an application, an international application PCT/US2007/025978, filed on 20 Dec. 2007 and published as WO/2008 079 248 (which is incorporated herein by reference in its entirety) has proposed an endoscopic device for welding sutures comprising a jaw defining an area for gripping at least two portions of suture to be welded together. The endoscopic suture welding device also comprises, inside the gripping area, an electric heating element used to weld portions of sutures by being applied against the latter. An international application PCT/US2007/025977, filed on 20 Dec. 2007 and published as WO/2008 079 247 (which is incorporated herein by reference in its entirety) has, moreover, proposed a heating element that can be used in such an endoscopic suture welding device. This heating element comprises a substrate produced in a dielectric or insulating material, such as a non-conducting ceramic or a polyimide. This substrate comprises, at the level of a working face, an adhesion layer on which is deposited a conductive layer defining electrical contacts linked by a thin resistive element made of gold. The working face covered in this way with these various layers may then be applied against the portions of suture that have to be welded. Such a heating element effectively makes it possible to produce sutures by welding within the context of the device described by the application PCT/US2007/025978.

Firstly, the dielectric substrate used may exhibit a certain mechanical fragility in that it may crack under the effect of the successive thermal shocks that it will experience when it is heated and applied against the portions of sutures to be welded. Furthermore, it may also crack under the effect of the mechanical stresses resulting from its application against the sutures. The thickness of the substrate may be increased, but then the latter has such a thickness that it may no longer be able to appropriately hug the shape of the suture strands to be welded, so that the effectiveness of the heat transfer by conduction is no longer assured. Thicker ceramic heaters may also cause the jaws to get bigger, because the heaters must often be kept at a fixed distance for the suture to fit. This may be characterized as a fit issue rather than a thermal transfer issue. Furthermore, in the context of the use of a thick dielectric substrate, the latter may exhibit a relatively high thermal inertia which slows down the suturing process inasmuch as the heating element will take a certain time to reach the required melting temperature. Moreover, inasmuch as, according to WO/2008 079 247, the resistive heating element is placed on the working face of the substrate to offset the thermal inertia of the latter and is then placed in contact with the portions of suture strands to be welded, which may result in gradual wear of the resistive heating element and of its associated connecting elements. Thus, as sutures are made, the quality of the latter is likely to vary and, after a certain usage time, the heating element may no longer be able to appropriately perform its function. Moreover, the high thermal inertia of the substrate may penalize the device and impose the use of a high-capacity electric energy source that is detrimental to its maneuverability. The system with the ceramic heater is less efficient than the systems proposed herein, but both systems in some cases use the same number of batteries. In some cases, the ceramic system uses more energy but not enough to be detrimental to maneuverability.

Embodiments of the present invention include a new type of heating element that makes it possible to remedy the drawbacks of the heating element according to the prior art, while offering a much lower manufacturing cost.

Some embodiments include a heating element with dimensions less than a centimeter, comprising:
a metallic substrate which comprises a material having a thermal conductivity greater than 5 W/m·K and an ultimate tensile strength measured at ambient temperature greater than 400 MPa and which comprises a working face suitable to be placed in contact with a medium or an element to be heated,
a conductive layer which is supported by a face of the substrate opposite the working face and which forms a circuit comprising two power supply elements linked by a thin resistive element and intended to be connected to a current source, and
a dielectric layer, interposed between the conductive layer and the metallic substrate.

In the context of present disclosure, the ultimate tensile strength corresponds to the mechanical strength traditionally designated U.T.S.

The use of a metallic substrate makes it possible to obtain good mechanical strength and thermal shock resistance characteristics for the heating element inasmuch as the metallic substrate ensures the cohesion of the assembly. Furthermore, the use of a metallic substrate makes it possible to use the latter as a thermal conduction element to handle the transfer of the heat produced by the resistive element to the medium or the element to be heated. Thus, in the context of a use of the heating element according to embodiments of the invention in a suture welding application, it is the working face which is in contact with the sutures to be welded, whereas the conductive layer is on the opposite face and is not therefore subject to the risk of abrasion that bringing it into repeated contact with the sutures to be heated could engender. Moreover, the use of a thin substrate makes it possible to reduce the thermal inertia of the heating element and obtain a substrate that has elastic deformation capabilities such that the working face of the substrate can hug, at least partly, the shape of the element to be heated which is placed in contact with it.

In order to optimize this elastic deformation capability, in a heating element according to one embodiment of the invention:
the metallic substrate has a thickness within a range of values extending from 2 μm to 1 mm and preferably from 2 μm to 0.5 mm ;
the dielectric layer has a thickness within a range of values extending from 2 μm to 25 μm and preferably from 2 μm to 4 μm ;
the portion of the conductive layer forming the thin resistive element has a thickness less than 2 μm ;
the portion of the conductive layer forming the link branches has a thickness within a range of values extending from 2 μm to 3 mm.

Thus, the use of such dimensional characteristics for the different constituent layers of the heating element makes it possible to obtain optimal thermal and mechanical characteristics. Furthermore, given the thickness values adopted, it is possible to use routine microelectronics manufacturing techniques including, but not limited to, electrodeposition techniques, ion deposition techniques, gas phase deposition techniques, plasma etching techniques, or even photolithography techniques, sputtering, or spin coating.

According to an alternative embodiment of the invention and according to the nature of the substrate and of the constituent materials of the conductive layer, an adhesion layer may be interposed between the dielectric layer and the conductive layer. Thus, according to a such alternative embodiment of the invention, the adhesion layer can then be produced in such a way as to have a thickness less than 0.1 μm, for example between 200 and 300 angstroms.

The realization of a resistive element can be obtained by microelectronic manufacturing techniques and makes it possible to perform, at low cost, an accurate adjustment of the electrical conductivity characteristics of said resistive element.

According to an alternative embodiment of the invention, the thin resistive element is configured to present a variable current passage section. The implementation of a variable current passage section makes it possible to modulate the local electrical resistance and therefore the power locally dissipated by the resistive element.

Thus, it is possible to produce the thin resistive element in such a way that the latter has a generally elongate form with a current passage section which decreases from the middle of the resistive element towards the areas of connection of the resistive elements to the power supply elements. Such a general configuration of the thin resistive element makes it possible to obtain a distribution of the heat at the level of the working face of the substrate that is substantially uniform, so that this working face will exhibit a surface temperature that is substantially equal at all points, which, in the context of the application to the welding of sutures guarantees the quality of the weld.

In the case of the use, for the production of the thin resistive element, of microelectronic deposition techniques, and when these deposition techniques do not make it possible to act easily on the deposited thickness, the thin resistive element may exhibit a constant thickness and a width that is variable so as to modulate the thermal power dissipated over the length of the thin resistive element, according to embodiments of the present invention.

As stated previously, the implementation of a metallic substrate, which exhibits good thermal conductivity or conductivity characteristics, or even mechanical strength characteristics, makes it possible to produce a heating element exhibiting a high thermal density at the level of its working face. Thus, the resistive element can be adapted to obtain, at the level of the working face of the substrate, a heat density greater than 3 W/mm². This embodiment of the thin resistive element and of the heating element makes it particularly suitable to the production of welded suture stitches.

In the same spirit, and according to another embodiment of the heating element, the different constituent materials of the heating element are adapted to withstand operating temperatures greater than 250° C. without impairing their mechanical properties.

According to embodiments of the present invention, different types of materials can be envisaged for the substrate and the various constituent layers of the heating element.

Thus, for example, the constituent material of the substrate can be chosen from:
a sheet of titanium alloy containing aluminium, niobium, vanadium or zirconium,
a sheet of titanium alloy Ti-3Al-2.5V
a sheet of stainless steel,
a sheet of pure titanium,
a strip obtained by stacking sheets chosen from the above sheets.

In the same way, for example, the constituent material of the insulating layer may be chosen from the following materials:
polyimide,
alumina,
glass,
polytetrafluoroethylene,
parylene,
silica,
zircon.

The constituent material of the thin resistive element can be chosen from the following materials, for example:
gold,
niobium,
palladium,
platinum,
tantalum,
titanium,
zirconium,
chromium,
nickel,
carbon,
alloys or mixtures of these materials.

The power supply elements may be produced using gold or copper covered with nickel.

Finally, the constituent material of the adhesion layer may be chosen from the following materials, for example:
alloy of titanium-tungsten,
pure titanium.

Obviously, these lists of materials are neither exhaustive nor limiting, inasmuch as any other suitable material could be used. In the context of medical applications, constituent materials of the heating elements may be chosen from biocompatible materials.

Embodiments of the present invention also relate to an endoscopic device for welding sutures comprising a jaw defining an area for gripping at least two portions of sutures to be welded together and a heating element according to an embodiment of the invention which is arranged in the gripping area and whose working face is oriented so as to be able to be applied against the portions to be welded.

Obviously, the different characteristics, forms and variants of embodiments of the invention can be associated with one another in various combinations, inasmuch as they are not mutually exclusive or incompatible.

Moreover, various other characteristics and benefits of the invention will emerge from the description given hereinbelow with reference to the appended drawings which illustrate a nonlimiting embodiment of a heating element and a device implementing such a heating element.

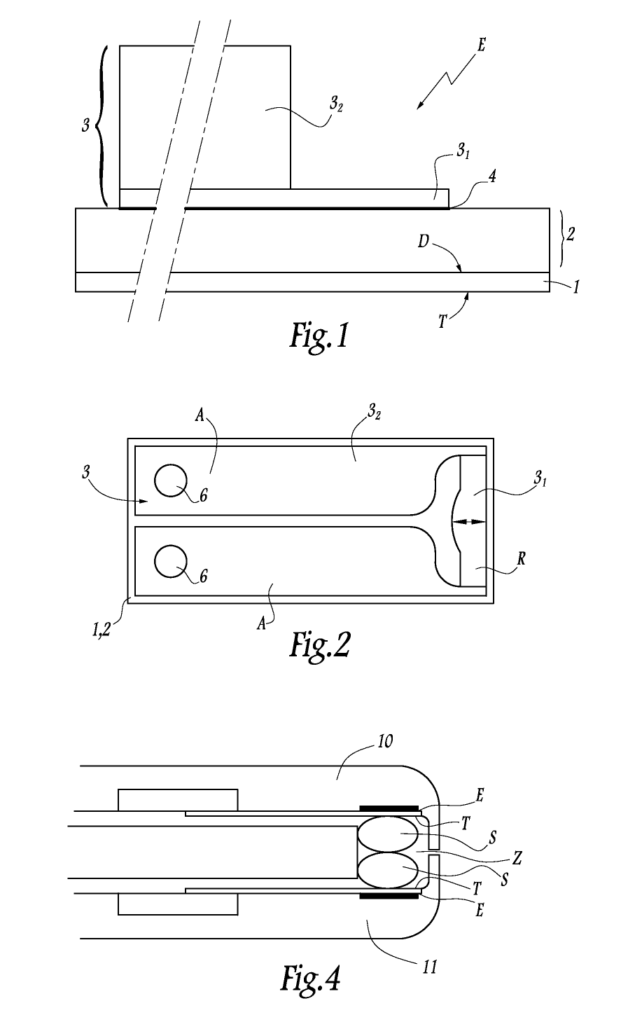

FIG. 1 is a diagrammatic transversal cross section showing the different constituent layers of a heating element according to embodiments of the invention.

FIG. 2 is a diagrammatic plan view showing a heating element according to an embodiment of the invention.

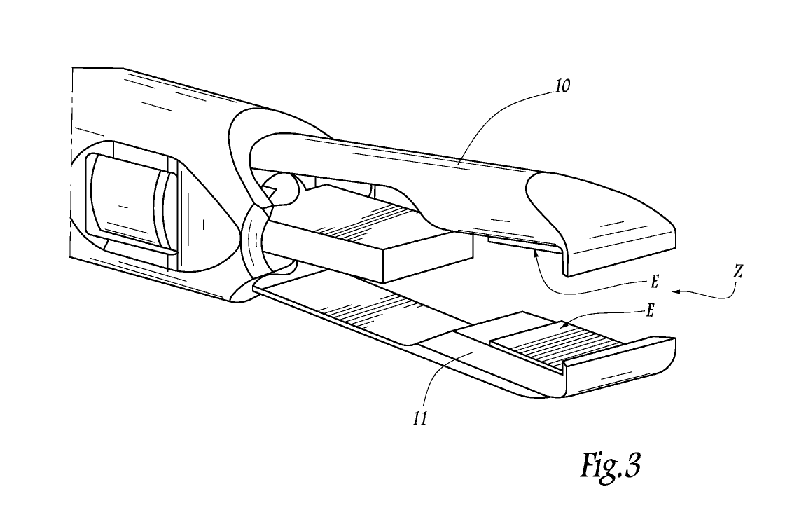

FIG. 3 is a diagrammatic perspective view of an endoscopic device for welding sutures.

FIG. 4 is a diagrammatic cross section of the jaw of the endoscopic device of FIG. 3 when making sutures by welding.

A heating element according to an embodiment of the invention, as illustrated in FIG. 1 and designated as a whole by the reference E, comprises a metallic substrate 1 which is made up of a material having a conductivity, also called thermal conductivity, greater than 5 W/m·K and an ultimate tensile strength measured at ambient temperature greater than 400 MPa. The metallic substrate is, for example, formed by a rollable sheet of a titanium alloy, it being possible, for example, for the substrate to be a sheet of titanium alloy of Ti 3Al 2.5V type having a thickness of several microns, for example 2 μm.

The substrate 1 can, for example, present a generally rectangular shape, as shown in FIG. 2.

The substrate 1 has a working face T intended to be placed in contact with the medium or the element to be heated. The working face T is preferably kept bare so as not to affect its thermal conductivity characteristics. On the other hand, the face D, called the deposition face, of the substrate, is situated on the opposite side to the working face T and is covered, at least partly, with a dielectric layer of any appropriate nature, for example from the family of polyimides. The dielectric layer 2 can be deposited on the deposition face D of the substrate 1 by any appropriate technique, such as, for example, but not exclusively, by means of a centrifugal deposition technique, also called “spin coating”. The dielectric layer can have a thickness of the order of several microns, such as, for example 2 μm to 4 μm. It should be noted that the dielectric layer can have a thickness close to or less than that of the substrate 1 or even a thickness greater than the latter. In this regard, it should be noted that FIG. 1 is a diagrammatic cross section showing the stacking of the different structural layers that make up the heating element E with the different thicknesses of the constituent layers of the heating element E not being drawn to scale.

The dielectric layer 2 bears, on the opposite side to the substrate 1, a conductive layer 3. Depending on the nature of the conductive layer 3 and of the dielectric layer 2, the heating element may include an adhesion layer 4 interposed between the dielectric layer 2 and the conductive layer 3. According to the example illustrated, the adhesion layer 4 is a layer of titanium alloy with a thickness less than 0.1 μm, deposited by any appropriate microelectronics technique, such as, for example, by sputtering of thin films.

The conductive layer 3 first comprises a first thickness of conductive material, intended to form a thin resistive element.

The first conductive thickness 3₁ is, for example, formed by a submicron layer of gold deposited by sputtering of thin films onto the adhesion layer 4. The conductive layer 3 also comprises a second thickness 3₂ which may form power supply elements for the resistive element R formed by the first thickness 3₁. Inasmuch as the two thicknesses of the conductive layer 3 are produced in the same material, the structure of the conductive layer 3 can be obtained by the conventional microelectronic techniques, such as, for example, photolithography or photogravure. Thus, the conductive layer 3 and the adhesion layer 4 can be structured in such a way as to exhibit a pattern as illustrated in FIG. 2, from which it emerges that the conductive layer comprises two power supply elements A formed, according to the example illustrated, by two parallel branches which are linked at their ends by the thin resistive element R. The power supply elements A also each present, on the opposite side to the resistive element R, an area 6 for receiving an electrical power supply wire. The two power supply elements A are produced in such a way as to have a thickness of several microns, for example of 5 um (electrodeposited gold) whereas the portion of the conductive layer that forms the thin resistive element R may have a thickness less than a micron and, for example, of the order of 0.25 um (sputtered gold).

Given the structure of the conductive layer 3, the power supply elements A have a low electrical resistance, whereas the thin resistive element R will have a high electrical resistance, so that the heating of the heating element E resulting from the joules effect will be concentrated in the region of the resistive element R. According to the example illustrated, the thin resistive element R has an elongate shape and, in order to modulate the thermal power available over its length, the thin resistive element R is structured so as to have a width, measured transversely to its length, that is greater at its centre and gradually decreases from the centre towards the ends linking to the power supply elements A, according to embodiments of the present invention. Thus, inasmuch as the thin resistive element has a constant thickness, its electrical resistance in its central region will be lower than its end regions linking to the power supply elements A. Thus, by modulating the width of the thin resistive element R, a uniform temperature of the working face T of the heating element E situated facing the resistive element R is obtained, in steady-state operation. The heating element R produced in this way can, given the nature of the materials selected, withstand operating temperatures greater than 250° C. and offer a thermal power density greater than 3 Watts/mm², which makes the heating element particularly suited to applications where it is necessary to have, rapidly, high temperatures that are uniformly distributed. Such is, for example, the case with welded suture applications performed endoscopically.

Thus, the heating element according to embodiments of the present invention is particularly suited to incorporation in an endoscopic suture welding device, as illustrated in FIG. 3. Such a device generally comprises a jaw formed by two beaks 10 and 11 which define an area Z for gripping at least two portions S of sutures to be welded. The endoscopic device then comprises at least one, and, according to the example illustrated, two, heating elements E according to the invention, which are each fitted to a jaw, so as to be placed facing one another. Thus, when the jaw is closed, the working faces T of the heating elements are applied against the portions S of sutures to be welded, as shown in FIG. 4, so as to ensure the melting thereof. The uniformly distributed high temperatures allowed by the heating elements E according to embodiments of the invention make it possible to obtain a very high quality suture weld. Moreover, inasmuch as the conductive layers are not placed directly in contact with the elements to be welded, they are not subject to a risk of wear by abrasion.

Thus, the heating element according to an embodiment of the invention makes it possible to obtain a particularly satisfactory operation of the endoscopic device which it equips.

Moreover, inasmuch as the heating element according to embodiments of the invention can be produced easily by microelectronics-oriented manufacturing techniques, its cost price is particularly low, so that it can be used in applications requiring controlled temperatures other than endoscopic welding, such as, for example, the applications of temperature stabilization of electronic components such as crystal oscillators, power supplies, amplifiers, accelerometers, digital-analogue converters, current sources or voltage sources, strain gauges, transistor circuits. The aforementioned list is in no way limiting or exhaustive.

According to the examples illustrated, the heating element has an adhesion layer 4. However, such an adhesion layer is optional and depends on the compatibility between the conductive layer and the insulating layer. Similarly, according to the example illustrated, the conductive layer 3 may be made of one and the same material, but it would also be possible to produce the thickness 3₂ forming the resistive element R in another material than the thickness 3₂ intended to form the power supply elements of the thin resistive element R.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims and the present disclosure, together with all equivalents thereof.

What is claimed is:

1. A heating element with dimensions less than a centimetre, comprising:
a metallic substrate (1) which comprises a material having a thermal conductivity greater than 5 W/m·k and an ultimate tensile strength measured at ambient temperature greater than 400 MPa and which comprises a working face suitable to be placed in contact with a medium or an element to be heated,
a conductive layer (3) which is supported by a face of the substrate opposite the working face and which forms a circuit comprising two power supply elements (A) linked by a thin resistive element (R) and adapted to be connected to a current source, and
a dielectric layer (2) interposed between the conductive layer (3) and the metallic substrate (1).

2. The heating element according to claim 1, characterized in that:
the metallic substrate (1) has a thickness within a range of values extending from 2 μm to 1 mm and preferably from 2 μm to 0.5 mm,
the dielectric layer (2) has a thickness within a range of values extending from 2 μm to 25 μm and, preferably, extending from 2 μm to 4 μm,
the portion (31) of the conductive layer forming the thin resistive element (R) has a thickness less than 2 μm;
the portion (32) of the conductive layer forming the link branches (A) has a thickness within a range of values extending from 2 μm to 3 mm.

3. The heating element according to either of claims 1 and 2, further comprising an adhesion layer (4) interposed between the dielectric layer (2) and the conductive layer (3).

4. The heating element according to claim 3, characterized in that the adhesion layer (4) has a thickness less than 0.1 μm.

5. The heating element according to one of claims 1 to 4, characterized in that the thin resistive element (R) is configured to present a variable current passage section.

6. The heating element according to one of claims 1 to 4, characterized in that the thin resistive element (R) is configured to have a generally elongate form with a current passage section which decreases from the middle of the resistive element towards the areas of connection of the resistive element to the power supply elements.

7. The heating element according to one of claims 1 to 6, characterized in that the thin resistive element (R) is adapted to obtain, at the level of the working face of the substrate, a heat density greater than 3 W/mm2.

8. The heating element according to one of claims 1 to 7, characterized in that the constituent material of the substrate (1) is chosen from:
(a) a sheet of titanium alloy containing one or more of aluminium, niobium, vanadium and zirconium,
(b) a sheet of titanium alloy Ti 3AI 2.5V,
(c) a sheet of stainless steel,
(d) a sheet of pure titanium,
(e) a strip obtained by stacking two or more sheets chosen from the sheets (a), (b), (c), and (d), above.

9. A surgical device for welding sutures comprising at least one heating element (E) according to one of the preceding claims, which is arranged in a gripping area (Z) and whose working face (T) is oriented so as to be able to be applied against portions (S) of suture to be welded together.

10. Surgical device according to claim 9, characterized in that the device comprises a jaw which defines the gripping area (Z) and to which two heating elements (E) facing one another are fitted.
